(19) United States
(12) Patent Application Publication
(10) Pub. No.: US 2009/0069638 A1
(43) Pub. Date: Mar. 12, 2009

(54) Diagnostic and Treatment Regimen for Achieving Body Water Homeostasis

(76) Inventor: Howard Murad, Marina Del Rey, CA (US)

Correspondence Address: LOUIS C. PAUL, 420 East 61st Street, 8E, NEW YORK, NY, 10021, US

(21) Appl. No.: 12/085,906

(22) PCT Filed: Dec. 4, 2006
(86) PCT No.: PCT/US06/45975

§ 371 Date: Jun. 2, 2008

Related U.S. Application Data
(60) Provisional application No. 60/741,448, filed on Dec. 2, 2005.

(51) Int. Cl. A61B 5/00; A61M 31/00

(52) U.S. Cl. 600/300; 604/500

(57) ABSTRACT

A method for achieving and maintaining an optimized cellular water homeostasis and good connective tissue health in humans, where cellular water homeostasis is expressed as the ratio of intercellular water (“ICW”) content to extracellular water (“ECW”) content homeostasis comprising the steps of (i) taking a first set of measurements of two or more of ICW, ECW and total body water (“TBW”) contend; (ii) comparing the first set of measurements against a predetermined set of values associated with a population of people; (iii) administering an initial dosing of a first composition comprising a therapeutically-effective amount of an amino acid/phospholipid pairing (“AAPP”); (iv) taking a follow-up set of measurements of two or more of TBW, ICW and/or ECW after the initial dosing; (v) comparing the follow-up set of measurements against the predetermined set of population values; (vi) administering a follow-up dosing of a follow-up composition comprising a therapeutically-effective amount of an AAPP; (vii) repeating steps (iv) through (vi) until the desired values of TBW, ICW and/or ECW are achieved.

### FIELD OF INVENTION

The present invention relates to a diagnostic and treatment regimen for dermatologic disorders as well as other disease conditions and states. More particularly, the regimen of the present invention is useful in reducing the signs and appearance of photoaging and biological aging.

### BACKGROUND OF THE INVENTION

A multitude of theories have been advanced to explain aging and disease processes. Senescence has been explained in terms of “programmed” cell death, somatic mutation and autoimmune reactions, and free-radical damage. The latter relates to cells' decreased ability to respond to and repair damage caused by oxygen free radical species. Free radicals are produced both during normal metabolic processes and in response to external factors (e.g., exposure to ultraviolet radiation). From a clinical dermatological perspective, they are associated, for example, with the formation of wrinkles and less supple skin.

Common to these theories is a recognition that cells undergo bio-chemical/bio-physical alterations in a manner that may compromise their functionality. Put differently, the aging process, internal and external environmental stressors, as well as disease, all affect the delicate homeostatic balance that keeps living cells functioning properly.

Water is the single most plenteous component of the human body. Accordingly, one of the most important measures of overall health is total body water (“TBW”) content, which can be further compartmentalized into intracellular water (“ICW”) content and extracellular water (“ECW”) content. As used in the present application, TBW is the sum of ICW and ECW.

Free radicals, as well as other sequelae of internal and external stressors, disease and aging, affect ICW/ECW homeostasis. More particularly, free radicals and other insults, alter, and in many instances, damage the cytoplasmic membranes that envelop and protect the components of the cells. This, in turn, affects ICW and ECW. The association of loss of intracellular water content and aging is described in the literature, including by Murad, The Cellulite Solution (St. Martin's Press, 2004) and Nagy, “A membrane hypothesis of aging,” J. Theor. Biol. 75: 189-195 (1978) and Nagy, “The role of membrane structure and function in cellular aging: a review” Mech. Ageing Dev. 9: 237-246 (1979). Whereas Nagy explains senescence in terms of water loss caused by cell membrane damage, the diagnostic and treatment regimen of the present invention is based on correcting suboptimal distribution and use of water in both intracellular and extracellular compartments (e.g., connective tissue).

Capacitors are electrical components that surround an electric charge with an insulating, non-conductive, layer—also referred to as a dielectric layer—to contain the charge. Capacitors are characterized both in terms of the amount of charge stored and the integrity and the ability of the dielectric layer to hold the stored charge. If the dielectric layer is weak or damaged, the stored charge drains away and/or the quantity of stored charge is less.

Similarly, the components of living cells are bathed in an ionic, charged fluid medium and are contained (i.e., enveloped) by a cytoplasmic membrane. This membrane, comprised of phospholipids, forms a dielectric layer around the ionic cytoplasm. When exposed to a high-frequency, low-level alternating current, there is bioelectrical impedance, a lag between applied voltage and measured electrical current. This lag is known as the phase angle.

Phase angle reflects the integrity of the capacitors formed by living cells and their cytoplasmic membranes. More particularly, phase angle is related to the integrity of the cytoplasmic membrane dielectric layer. Cells with intact membranes act as better capacitors, have higher capacitive reactance and, thus, higher measured phase angle values. Conversely, cells with a lower degree of cytoplasmic membrane integrity have a lower capacitance and demonstrate lower or decreased phase angle value. The use of phase angle in the healing arts is reported in the literature. It has been used both to diagnose disease and to demonstrate improvements in cytoplasmic membrane integrity as a result of therapeutic intervention.

The present invention is an improvement of pending U.S. patent application Ser. No. 11/090,567, now Publication No. 2005/0261367, the disclosure of which is incorporated herein by reference. There remains a long-felt, but as yet unmet, need for improving cytoplasmic membrane integrity and thus, achieving and maintaining, an optimized ICW/ECW homeostasis and good connective tissue health. The treatment regimen of the present invention, which comprises administering compositions comprising a therapeutically-effective amount of an amino acid/phospholipid pairing (AAPP), both alone and in combination with one or more of an essential fatty acid, a glycosaminoglycan or glucosamine, an antioxidant, an anti-inflammatory agent, a mineral or vitamin, meets this heretofore unmet need.

### SUMMARY OF THE INVENTION

The present invention relates to a novel method for achieving and maintaining an optimized ICW/ECW homeostasis and connective tissue health in humans comprising the steps of (i) taking a first set of measurements of two or more of TBW, ICW and/or ECW; (ii) comparing the first measurements against a predetermined set of values associated with a population of people; (iii) administering an initial dosing of a first composition comprising a therapeutically-effective amount of an AAPP; (iv) taking a follow-up set of measurements of two or more of TBW, ICW and/or ECW after the initial dosing; (v) comparing the follow-up measurements against the predetermined set of population values; (vi) administering a follow-up dosing of a follow-up composition comprising a therapeutically-effective amount of an AAPP; (vii) repeating steps (iv) through (vi) until the desired values of TBW, ICW and/or ECW are achieved.

### DETAILED DESCRIPTION OF THE INVENTION

The present invention relates to a novel method for achieving and maintaining an optimized ICW/ECW homeostasis and connective tissue health in humans comprising the steps of (i) taking a first set of measurements of two or more of TBW, ICW and/or ECW; (ii) comparing the first measurements against a predetermined set of values associated with a population of people; (iii) administering an initial dosing of a first composition comprising a therapeutically-effective amount of an AAPP; (iv) taking a follow-up set of measurements of two or more of TBW, ICW and/or ECW after the initial dosing; (v) comparing the follow-up measurements against the predetermined set of population values; (vi) administering a follow-up dosing of a follow-up composition comprising a therapeutically-effective amount of an AAPP; (vii) repeating steps (iv) through (vi) until the desired values of TBW, ICW and/or ECW are achieved. As used in the present invention, the phrase “therapeutically-effective amount” means an amount of an AAPP that provides the desired therapeutic benefit generally and particularly in terms of improving and maintaining TBW, ICW and/or ECW.

In adults (i.e., persons above 18 years of age), TBW can range from about 45% to about 60% of total body weight. For purposes of the present invention, TBW can be stratified into four quartiles indicating the degree of cell water homeostasis of an individual patient compared to a predetermined set of average values associated with a population of people. According to the treatment regimen of the present invention, persons with TBW of less than about 48 would be considered clinically as “diseased”. Persons with TBW of from about 48 to about 52 are considered to be “below average”. Persons with TBW of from about 53 to about 56 are considered to be “average”. Persons with TBW of from about 57 to about 60 are considered to be “above average”.

According to one aspect of the present invention, one or more compositions comprising a therapeutically-effective amount of an MPFI are administered in one or more dosings until an average TBW (i.e., based on gender and age) is achieved and maintained. More particularly, after taking an initial set of measurements of two or more of TBW, ICW and/or ECW, and comparing those values to a predetermined set of population values, a patient receives an initial dosing of a first composition comprising a therapeutically-effective amount of an AAPP. Depending on the values of ICW and ECW, the first composition may comprise, in addition to the AAPP, one or more of an essential fatty acid, a glycosaminoglycan or glucosamine, an antioxidant, an anti-inflammatory agent, a trace mineral or vitamin.

A follow-up set of measurements of two or more of TBW, IOW and/or ECW is taken after the initial dosing and compared against the predetermined set of population values. A follow-up dosing of a follow-up composition comprising a therapeutically-effective amount of an AAPP is then administered. In one embodiment, the follow-up composition may comprise the first composition and a supplemental “booster” pack. The booster pack may comprise additional amounts of one or more amino acids, additional amounts of one or more phospholipids, or both. Separately, or in addition to the increased amounts of amino acid(s) and/or phospholipid(s), the booster pack may comprise one or more of an essential fatty acid, a glycosaminoglycan or glucosamine, an antioxidant, an anti-inflammatory agent, a trace mineral or vitamin.

Measurements of two or more of TBW, ICW and/or ICW are again taken. These steps are repeated until an “average” ICW and/or ECW is achieved. In one aspect of the present invention, when an average ICW is achieved, the follow-up composition is not changed. Two or more of the patient's TBW, ICW and/or ECW are measured thereafter to confirm that an “average” IGW and/or ECW is being maintained.

In a preferred embodiment, one or more compositions comprisicg a therapeutically-effective amount of an AAPP are administered in one or more dosings until an “above average” ICW and/or ECW is achieved and maintained. According to one aspect of the present invention, where a patient begins with an “average” ICW and/or ECW and achieves an “above average” ICW and/or ECW after the initial dosing, the follow-up composition is the same as the first composition.

For men ages 18 to 65 years of age, ICW constitutes from about 60% to about 67% of TBW (median of 63.5%); ECW constitutes from about 33% to about 40% of TBW (median of 36.5%). For men ages 66 to 80, ICW constitutes from about 52% to about 58% of TBW (median of 55%); ECW constitutes from about 42% to about 48% of TBW (median of 45%) In women ages 18 to 65, ICW constitutes from about 55% to about 58% of TBW (median of 56.5%); ECW constitutes from about 42% to about 45% of TBW (median of 43.5%). For women 66 to 80 years of age, ICW constitutes from about 48% to about 53% of TBW (median of 50.5%); ECW constitutes from about 47% to about 52% of TBW (median of 49.5%).

ICW and ECW, like TBW, can be stratified into four quartiles, with age- and gender-specific subdivisions, indicating the degree of ICW/ECW homeostasis compared to a predetermined set of average values associated with a population of people. Other stratifications are possible including, without limitation, those based on height and weight. For purposes of the present investigation, stratifications based on one or more of age, gender, height and/or weight are referred to as “AGHW” optimized values. ICW ranges, based on the median percentage values for ICW and expressed as percent of body weight, stratified by both gender and age, are displayed below:

| | Men | Men | Women | Women |
|---|---|---|---|---|
| | (18-65) | (66-80) | (18-65) | (66-80) |
| Diseased | <30.5 | <26.5 | <27 | <24 |
| Below Average | 30.5-33.0 | 26.5-28.5 | 27.0-29.5 | 24.0-26.0 |
| Average | 33.5-35.5 | 29.0-31.0 | 30.0-31.5 | 26.5-28.0 |
| Above Average | 36.0-38.0 | 31.5-33.0 | 32.0-34.0 | 28.5-30.5 |

According to one aspect of the present invention, one or more compositions an AAPP are administered in one or more dosings until an average IGW (e.g., based on gender and age) is achieved and maintained. In a preferred embodiment, one or more compositions are administered in one or more dosings until an above average ICW is achieved and maintained.

TBW, ICW and ECW can be measured and analyzed using bioelectric impedance analysis, referred to hereinbelow as BIA. More particularly, BIA measures body resistance and capacitive reactance when a low level, high-frequency alternating current is passed through the body (i.e., via electrodes). In turn, body resistance and capacitive reactance allow the computation of phase angle—mathematically, a function of the arctangent of the ratio of reactance over resistance. BIA devices suitable for use in the present invention are commercially available, including from RJL Systems (Clinton Twp., MI).

Without wishing to be bound to a theory, applicant believes that ICW correlates generally to the number of healthy cells, and more particularly correlates with the integrity of the cells' cytoplasmic membranes.

In many pathophysiological conditions, TBW (and, in turn, ICW and ECW) are outside of the AGHW-optimized ranges. Where a patient presents with elevated TBW, the cause may be due to either ECW or ICW. For example, ECW may be elevated and (CW may be low. In such a case, the patient's TBW would include “wasted” water. As used in the present application, “wasted water” means water outside cells and outside connective tissue.

Without wishing to be bound to a theory, applicant believes that one cause of elevated TBW where ECW is higher than the AGHW optimized values and ICW is lower than the AGHW-optimized values may be an increased number of damaged or sub-optimally functioning cytoplasmic membranes. Causes of cytoplasmic membrane damage/dysfunction may include free radicals, inflammation, stress, microbes, as well as stressors, both internal and external. Clinically, patients with comparatively higher ECW and ICW lower (i.e., than the AGHW-optimized values) present with puffiness, bloating.

Elevated TBW can also occur where ECW is low and ICW is high (both in comparison to their respective AGHW-optimized ranges). Conditions in which this is believed to occur include connective tissue damage. The term “connective tissue” as used in the present application means tissue that has very few cells and is predominantly made up of fibrous material, such as the proteins collagens and elastins as well as other glycosaminoglycans. Non-limiting examples of connective tissue include the dermis, blood vessels, nerves, tendons and ligaments. The phrase “connective tissue disease” as used in the present application is defined as a disorder, hereditary or acquired, characterized by abnormal structure or function of one or more of the elements of connective tissue, e.g., collagen, elastin, or the mucopolysaccharides. Examples of connective tissue diseases are listed in US Patent Application Publication No. 2005/0129787, the disclosure of which is incorporated herein by reference in its entirety. “Connective tissue health” as used in the present application is defined as a non-diseased condition or state characterized by normal structure and functioning of collagen, elastin, mucopolysaccharides as well as other connective tissues. Persons with “good connective tissue health” have “average” or “above average” ECW compared to AGHW-optimized ranges.

One object of the present invention is to restore ICW and ECW to target AGHW-optimized ranges, either “average” or “above average”, and thus improve cytoplasmic membrane integrity and/or connective tissue health. These objectives are accomplished by a regimen of administering specific combinations of active ingredients.

Another object of the present invention is to increase phase angle, preferably by at least about 1%, more preferably by at least about 2%, still more preferably by at least about 5%, and even more preferably by at least about 10%.

In one embodiment, the initial dosing is a first composition comprised of (i) at least one phospholipid selected from the group consisting of lecithin and phosphatidylcholine and (ii) at least one amino acid is selected from the group consisting of phenylalanine, valine, tryptophan, tyrosine, isoleucine, methionine, histidine, alanine, leucine, lysine, proline, cysteine, glycine and glutamic acid. In a preferred embodiment, the at least one amino acid is selected from the group consisting of methionine, lysine, proline, cysteine and glycine.

In another preferred embodiment, choline is administered together with lecithin or phosphatidylcholine and at least one amino acid.

In a more preferred embodiment, the therapeutically-effective AAPP comprises at least one phospholipid and at least two amino acids. In a still more embodiment, the at least two amino acids are selected from the group consisting of methionine, lysine, proline, cysteine and glycine.

In an particularly preferred embodiment, the therapeutically-effective AAPP comprises (i) lecithin or phosphatidylcholine; (ii) choline; and (iii) at least two amino acids. In an even more preferred embodiment the at least two amino acids are selected from the group consisting of methionine, lysine, proline, cysteine, and glycine.

In other embodiments of the present invention, depending on whether the values of ICW and ECW are high or low in comparison to the AGHW population values, the therapeutically-effective AAPP is supplemented with one or more of an essential fatty acid (“EFA”), a glycosaminoglycan or glucosamine, an antioxidant, an anti-inflammatory agent, a trace mineral and/or a vitamin.

In one aspect of the invention, a therapeutically-effective amount of an AAPP is administered with a glycosaminoglycan. Preferred glycosaminoglycans suitable for use in the present invention are selected from the group consisting of hyaluronic acid, chondroitin, as well as their pharmaceutically acceptable salts and esters. A particularly preferred chondroitin is chondroitin sulfate.

In another aspect of the invention, a therapeutically-effective amount of an AAPP is administered with a glucosamine, or one of its pharmaceutically acceptable salts or esters. A particularly preferred glucosamine is n-acetyl glucosamine.

In another aspect of the invention, a therapeutically-effective amount of an AAPP is administered with an EFA. Preferred EFAs suitable for use in the present invention are unsaturated C₁₈ fatty acids. Particularly preferred EFAs are selected from the group consisting of octadecenoic acids octadecadienoic acids and octadecatrienoic acids. Most preferred EFAs are selected from the group consisting of 9-octadecenoic acid; cis-cis-9,12-octadecadienoic acid; all-cis-9,12,15-octadecatrienoic acid; and all-cis-6,9,12-octadecatrienoic acid.

Sources of all-cis-9,12,15-octadecatrienoic acid include flax seed oil, canola oil and soybean oil. Sources of all-cis-6,9,12-octadecatrienoic acid are black currant oil, evening primrose oil, and borage oil. Sources of cis-cis-9,12-octadecadienoic acid and cis-9-octadecenoic acid are, respectively grapeseed oil and olive oil.

In another aspect of the present invention, a therapeutically-effective amount of an AAPP is administered with a glycosaminoglycan and/or glucosamine and an EFA. The foregoing combinations of a glycosaminoglycan and/or glucosamine and an EFA may be in the first composition administered or in a follow-up composition, including, but not limited to a booster pack.

In another embodiment of the present invention, a first composition or a follow-up composition may comprise an antioxidant. Non-limiting examples of antioxidants suitable for use in the present invention include: retinoids selected from the group consisting of retinol, retinal, retinol esters, retinyl propionate, retinoic acid and retinyl palmitate; ascorbic acid, derivatives of ascorbic acid and mixtures thereof; tocopherol, derivatives of tocopheroi and mixtures thereof; superoxide dismutase; polyphenols selected from the group consisting of phenolic acids, flavonoids, stilbenes and lignans; carotenoid selected from the group consisting of beta-carotene, alpha-carotene, luteifl, zeaxanthin, lycopene, and cryptoxanthin; botanical extracts selected from the group consisting of Punica granatum, Lycium barbarum, Morinda citrifolia, and Durio zibethinus; sulfhydryl compounds, including glutathione; Coenzyme Q10, derivatives of Coenzyme Q10, including hydroxydecyl ubiquinone, and mixtures thereof; silymarin. Particularly preferred antioxidants are retinoids, ascorbic acid and its derivatives, tocopherol and its derivatives and extracts of Punica granatum and Lycium barbarum.

In another embodiment of the present invention, a first composition or a follow-up composition may comprise an anti-inflammatory agent. The anti-inflammatory agent may be steroidal or non-steroidal. Pharmaceutically active non-steroidal anti-inflammatory agents suitable for use in the present invention may be selected from the group consisting of oxicams, salicylates, acetic acid derivatives, fenamates, propionic acid derivatives and pyrazoles. Additional non-steroidal anti-inflammatory agents suitable for use in the present invention include: allantoin; aloe vera; extracts of Arnica Montana, Chamomila recutita, Glycyrrhiza glabra or Lycium barbarum; and zinc. Preferred anti-inflammatory agents are allantoin and extract of Lycium barbarum.

Steroidal anti-inflammatory agent suitable for use in the present invention include: hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

In another embodiment of the present invention, a first composition or a follow-up composition may comprise a trace mineral and/or a vitamin. Preferred trace minerals suitable for use in the present invention are selected from the group consisting of magnesium, iron, zinc, copper, manganese, iodine, chromium, molybdenum, selenium. Preferred vitamins suitable for use in the present invention—in addition to Vitamins A, C and E—are B-complex vitamins, Vitamin F and derivatives thereof.

In still further aspects of the present invention, a therapeutically-effective amount of an AAPP may be administered in the following ingredient combinations, either in a first composition or in a follow-up composition: (i) AAPP+glycosaminoglycan or glucosamine+antioxidant; (ii) AAPP+glycosaminoglycan (or glucosamine)+anti-inflammatory agent; (iii) AAPP+glycosaminoglycan (or glucosamine)+antioxidant+anti-inflammatory agent; (iv) AAPP+EFA+antioxidant; (v) AAPP+EFA+anti-inflammatory agent; (vi) AAPP+EFA+antioxidant+anti-inflammatory agent; (vii) AAPP+glycosaminoglycan (or glucosamine)+EFA+antioxidant; (viii) AAPP+glycosaminoglycan (or glucosamine)+EFA+anti-inflammatory agent; (ix) AAPP+glycosaminoglycan (or glucosamine)+EFA+antioxidant+anti-inflammatory agent. Each of combinations (i) through (ix) may also be administered with one or both of a trace mineral and/or a vitamin as disclosed above.

Compositions of the present invention may be administered in a number of dosage forms and/or routes of administration including oral, topical, transdermal, subcutaneous, intramuscular, parenteral and intravenous. Preferred dosage forms are oral and topical. The ingredients used in the methods and compositions of the present invention may be administered individually, as a single composition that contains all the ingredients, or in combinations thereof. For example, the first composition may be comprised of separate caplets or a cream. Likewise, the first composition may be a unitary solid oral dosage form and may be administered in combination with a booster pack consisting of multiple capsules. As will be appreciated by persons of ordinary skill in the art, dose and dose frequency will vary among patients to account for, among other factors, age, weight, severity of condition(s) being treated.

The International Cosmetic Ingredient Dictionary and Handbook published by the Cosmetic, Toiletries & Fragrance Association describes a wide variety of non-limiting cosmetic and pharmaceutical ingredients that can help improve and maintain TBW, ICW and/or ECW to desired levels. Non-limiting examples of these ingredients include natural moisturizing factors (e.g., ceramides, triglycerides, glycosphingolipids, glycerin, sodium PCA), emollients, humectants, moisturizers and film formers or materials (e.g., polymers, for aiding the film-forming properties and substantivity of the composition). Other examples of cosmetic and/or pharmaceutical ingredients which are suitable for use in the delivery system of the present invention are disclosed in U.S. Pat. No. 6,492,326.

In another aspect of the present invention, where the patient is diagnosed with a disease, the AAPP (optionally supplemented with one or more of an antioxidant, an anti-inflammatory agent, an EFA, a glycosaminoglycan or glucosamine, a trace mineral, or a vitamin) may be administered in combination with a pharmaceutically active ingredient known to those of skill in the art as one that may be effective in treating the diagnosed disease. For example, a patient may be diagnosed with a suboptimal cell water condition characterized by a below average ICW and a microbial infection. In addition to the composition comprising a therapeutically-effective amount of an AAPP and an EFA, that patient may also receive, within the scope of the present invention, an appropriate pharmaceutically active ingredient, namely an antibiotic. Similarly, a hypertensive patient with below average ECW may receive both a composition comprising a therapeutically-effective amount of an APP, a glycosaminoglycan, and an appropriate pharmaceutically active ingredient, such as a vasodilator or a diuretic.

The following examples are further illustrative of the present invention. The components and specific ingredients are presented as being typical, and various modifications can be derived in view of the foregoing disclosure within the scope of the invention. In both examples, expected results are presented.

### EXAMPLE 1

Patient X, a forty-five year old male, visits a physician. He presents with an “above average” TBW (60% of body weight), where ICW is “below average” (31% of body weight) and ECW is “above average” (21.5% of body weight). Without wishing to be bound to a theory, applicant believes that this suboptimal cellular water state is indicative of cytoplasmic membrane damage and/or dysfunction. A starter kit comprising three dose packs—the first comprising a therapeutically-effective amount of an AAPP; a second comprising essential fatty acids; and a third comprising vitamins and trace minerals—is dispensed and is taken by the patient two times per day for four weeks. The contents of each dose pack are as follows:

Amino Acid and Phospholipid Dose
Pack (mg unless otherwise stated)

| | |
|---|---|
| L-Alanine | 100 |
| L-Arginine | 150 |
| L-Carnitine | 40 |
| L-Glycine | 75 |
| L-Histidine | 400 |
| L-Isoleucine | 400 |
| L-Leucine | 500 |
| L-Lysine | 250 |
| L-Proline | 500 |
| L-Selenomethionine | 80 μg |
| L-Taurine | 300 |
| L-Threonine | 150 |
| L-Tryptophan | 50 |
| L-Valine | 250 |
| N-Acetyl cysteine | 90 |
| Arachidonic acid | 75 |
| Choline | 75 |
| Inositol | 45 |
| Lecithin | 75 |
| Phosphatidic acid | 75 |
| Phosphatidyl choline | 45 |

Vitamin and Mineral Dose Pack (mg unless otherwise stated)

| | | |
|---|---|---|
| Beta carotene | 2500 | IU |
| Biotin | 300 | μg |
| Calcium carbonate | 60 | |
| Chromium picolinate | 15 | μg |
| Cupric oxide | 1000 | μg |
| Folic Acid | 400 | μg |
| Magnesium oxide | 200 | |
| Manganese | 2 | |
| Potassium iodide | 60 | |
| Sodium selenate | 50 | μg |
| Vitamin A acetate | 4000 | IU |
| Vitamin B-1 (Thiamine) | 25 | |
| Vitamin B-2 (Riboflavin) | 25 | |
| Vitamin B-3 (Niacinamide) | 60 | |
| Vitamin B-5 (Pantothenic acid) | 25 | |
| Vitamin B-6 | 20 | |
| Vitamin B-12 | 6 | μg |
| Vitamin C (Ascorbic acid) | 120 | |
| Vitamin E (DL-alpha tocopherol acetate) | 60 | IU |
| Zinc oxide | 15 | |

Essential Fatty Acid Dose Pack (mg unless otherwise stated)

| | | |
|---|---|---|
| Oleic Acid | 50 | 18:1 |
| Linoleic Acid | 50 | 18:2 |
| Alpha-Linolenic Acid | 50 | 18:3 |
| Gamma-Linolenic Acid | 50 | 18:3 |
| Stearidonic Acid | 50 | 18:4 |
| Dihomo-Gamma-Linolenic Acid | 50 | 20:3 |
| Arachidonic Acid | 100 | 20:4 |
| Eicosapentaenoic Acid | 50 | 20:5 |
| Docosahexaenoic Acid | 50 | 22:6 |

At the end of the four-week initial dosing, the patient returns to the doctor's office and a follow-up set of BIA measurements is taken. The follow-up series of measurements indicates some improvement of ICW (33% of body weight). A follow-up dosing is prescribed in which the starter kit is supplemented with a booster pack consisting of additional EFAs, together with a mixture of antioxidants and anti-inflammatory agents. The contents of the booster pack are as follows:

Booster Pack - Anti-inflammatory; Antioxidant;
EFAs (mg unless otherwise stated)

| | | |
|---|---|---|
| Borage oil | EFA | 250.0 |
| Flaxseed oil | EFA | 250.0 |
| Co-enzyme Q10 | Antioxidant | 1.5 |
| Fish oil | EFA | 450.0 |
| Extract of grape seed | Antioxidant | 10.0 |
| Extract of green tea | Antioxidant | 150.0 |
| Extract of Lycium barbarum | Anti-inflammatory | 20.0 |
| Extract of Punica granatum | Anti-inflammatory | 15.0 |

At the end of a second four-week period—in which the patient takes both the starter kit and the booster pack twice per day, each day of the four-week period—BIA measurements are taken and an “average” ICW is achieved (34.3% of body weight). The patient continues taking the starter pack and booster pack at the same dosing frequency for a three-month period at which time he returns to the physician's office for a maintenance visit. ICW (34.7% of body weight), ECW (20% of body weight) and TBW (54% of body weight) are measured and are all found to be “average”.

### EXAMPLE 2

Medispa Customer Y is a fifty year old female. At her initial wellness visit at the spa, she is found to have an “above average” TBW (58% of body weight), where ICW is “above average” (33.5% of body weight) and ECW is “below average” (21% of body weight). Without wishing to be bound to a theory, applicant believes that this customer's suboptimal cellular water state is indicative of poor connective tissue health. A one-month starter supplement kit comprising four daily dose packs for each day of the month is purchased. Two packs—the first comprising a therapeutically-effective amount of an AAPP and the second comprising essential fatty acids—have the same ingredients as described in Example 1. The third and fourth supplement packs comriprise, respectively, glycosaminoglycans and antioxidants, and have the following ingredients:

Glycosaminoglycan Pack (mg unless otherwise stated)

| | |
|---|---|
| Glucosamine sulfate | 1200 |
| N-Acetyl glucosamine | 160 |

Antioxidant Pack (mg unless otherwise stated)

| | | |
|---|---|---|
| Beta Carotene | 2500.0 | IU |
| Co-enzyme Q10 | 1.5 | |
| Extract of grape seed | 10.0 | |
| Extract of green tea | 150.0 | |
| Selenium | 50.0 | μg |
| Vitamin A | 4500.0 | IU |
| Vitamin C (ascorbic acid) | 300.0 | |
| Vitamin E (DL-alpha tocopherol acetate) | 60.0 | IU |

At her follow-up visit at the medispa facility, some improvement in “average ECW” (23.2% of body weight) is observed. Customer Y purchases a six-week supply of the starter supplement kit. At the end of the tenth week using the supplement kit, Customer V's values are measured and are “average”—ICW (30% of body weight), ECW (23.9% of body weight) and TBW (55% of body weight).

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A method for achieving and maintaining an optimized ICW/ECW homeostasis and good connective tissue health in humans comprising the steps of (i) taking a first set of measurements of two or more of TBW, ICW and/or ECW; (ii) comparing the first set of measurements against a predetermined set of values associated with a population of people; (iii) administering an initial dosing of a first composition comprising a therapeutically-effective amount of an amino acid/phospholipid pairing (“AAPP”); (iv) taking a follow-up set of measurements of two or more of TBW, ICW and/or ECW after the initial dosing; (v) comparing the follow-up set of measurements against the predetermined set of population values; (vi) administering a follow-up dosing of a follow-up composition comprising a therapeutically-effective amount of an AAPP; (vii) repeating steps (iv) through (vi) until the desired values of TBW, ICW and/or ECW are achieved.

2. The method of claim 1 wherein the initial dosing comprises an AAPP and at least one of an antioxidant, an anti-inflammatory agent, an essential fatty acid, a glycosaminoglycan, a trace mineral or a vitamin.

3-23. (canceled)

24. The method of claim 1 wherein the follow-up dosing comprises an AAPP and at least one of an antioxidant, an anti-inflammatory agent, an essential fatty acid, a glycosaminoglycan, a trace mineral or a vitamin.

25-45. (canceled)

46. The method of claim 1 wherein the AAPP in the initial dosing comprises an amino acid selected from the group consisting of phenylalanine, valine, tryptophan, tyrosine, isoleucine, methionine, histidine, alanine, leucine, lysine, proline, cysteine, glycine and glutamic acid.

47-49. (canceled)

50. The method of claim 1 wherein the AAPP in the initial dosing comprises a phospholipid selected from the group of lecithin and phosphatidylcholine.

51. The method of claim 50 wherein the AAPP in the initial dosing further comprises choline.

52-55. (canceled)

56. The method of claim 1 wherein the AAPP in the follow-up dosing comprises an amino acid selected from the group consisting of phenylalanine, valine, tryptophan, tyrosine, isoleucine, methionine, histidine, alanine, leucine, lysine, proline, cysteine, glycine and glutamic acid.

57-59. (canceled)

60. The method of claim 1 wherein the AAPP in the follow-up dosing comprises a phospholipid selected from the group of lecithin and phosphatidylcholine.

61. The method of claim 60 wherein the AAPP in the follow-up dosing further comprises choline.

62-65. (canceled)

66. The method of claim 2 where the essential fatty acid is an unsaturated C₁₈ fatty acid.

67. The method of claim 66 where the essential fatty acid is selected from the group consisting of octadecenoic acids, octadecadienoic acids and octadecatrienoic acids.

68. The method of claim 67 where the essential fatty acid is selected from the group consisting of 9-octadecenoic acid; cis-cis-9,12-octadecadienoic acid; all-cis-9,12,15-octadecatrienoic acid; and all-cis-6,9,12-octadecatrienoic acid.

69. The method of claim 24 where the essential fatty acid is an unsaturated C₁₈ fatty acid.

70-71. (canceled)

72. The method of claim 2 wherein the initial dosing comprises an AAPP and at least one glycosaminoglycan and the glycosaminoglycan is selected from the group consisting of hyaluronic acid, chondroitin, as well as their pharmaceutically acceptable salts and esters.

73. The method of claim 1 where the initial dosing comprises an AAPP and glucosamine.

74. The method of claim 73 where the glucosamine is n-acetyl glucosamine.

75. The method of claim 24 where the glycosaminoglycan is selected from the group consisting of hyaluronic acid, chondroitin, as well as their pharmaceutically acceptable salts and esters.

76. The method of claim 1 where the follow-up dosing comprises an AAPP and glucosamine.

77. The method of claim 76 where the glucosamine is n-acetyl glucosamine.

78. The method of claim 1 where according to the first set of measurements ICW is below average and the initial dosing comprises an AAPP and at least one essential fatty acid.

79. The method of claim 1 where according to the follow-up set of measurements ICW is below average and (i) the initial dosing comprises an AAPP and at least one essential fatty acid and the follow-up dosing comprises an amount of essential fatty acid greater than the amount in the initial dosing or (ii) the initial dosing does not contain an essential fatty acid and the follow-up dosing comprises at least one essential fatty acid.

80. The method of claim 1 where according to the first set of measurements ECW is below average and the initial dosing comprises an AAPP and at least one glycosaminoglycan.

81. The method of claim 1 where according to the follow-up set of measurements ECW is below average and (i) the initial dosing comprises an AAPP and at least one glycosaminoglycan and the follow-up dosing comprises an amount of glycosaminoglycan greater than the amount in the initial dosing or (ii) the initial dosing does not contain a glycosaminoglycan and the follow-up dosing comprises at least one glycosaminoglycan.

82. The method of claim 1 where the desired level of TBW is average.

83. The method of claim 1 where the desired level of TBW is above average.

84. The method of claim 1 where the desired level of ICW is average.

85. The method of claim 1 where the desired level of ICW is above average.

86. The method of claim 1 where TBW, ICW and/or ECW are measured by a licensed health care provider, an allied-health professional, or a trained technician.

87. The method of claim 1 where TBW, ICW and/or ECW are measured by an end-user or consumer.

88. The method of claim 1 where TBW, ICW and/or ECW are measured in a medical office or facility, an allied-health office or facility, a spa, a fitness or wellness facility, or a pharmacy.

89. The method of claim 1 where TBW, ICW and/or ECW are measured in a retail store.

90. The method of claim 1 where TBW, ICW and/or ECW are measured at home.

91. A method for increasing the phase angle in a person comprising administering therapeutically-effective amounts of an AAPP and at least one of an antioxidant, an anti-inflammatory agent, an essential fatty acid, a glycosaminoglycan, a trace mineral or a vitamin.

92-104. (canceled)

105. The method of claim 91 wherein the phase angle is increased by at least about 1%.

106. The method of claim 91 wherein the phase angle is increased by at least about 2%.

107. The method of claim 91 wherein the phase angle is increased by at least about 5%.

108. The method of claim 91 wherein the phase angle is increased by at least about 10%.
